(19) United States
(12) Patent Application Publication
(10) Pub. No.: US 2014/0343427 A1
(43) Pub. Date: Nov. 20, 2014

(54) DIAGNOSTIC IMAGING APPARATUS AND IMAGE DISPLAY METHOD

(71) Applicant: HITACHI MEDICAL CORPORATION, Tokyo (JP)

(72) Inventors: Syunya Fukunaga, Tokyo (JP); Osamu Mori, Tokyo (JP); Hirotsugu Yamada, Naruto-shi (JP)

(21) Appl. No.: 14/370,349

(22) PCT Filed: Jan. 9, 2013
(86) PCT No.: PCT/JP2013/050166

§ 371 Date: Jul. 2, 2014

(30) Foreign Application Priority Data
Jan. 12, 2012 (JP) 2012-003991

(51) Int. Cl. A61B 8/08; A61B 5/0205; A61B 5/044; A61B 5/11; A61B 8/00; A61B 8/14

(52) U.S. Cl. CPC A61B 8/5284; A61B 8/463; A61B 8/14; A61B 8/5223; A61B 8/54; A61B 8/5207; A61B 5/044; A61B 5/11; A61B 8/0883; A61B 5/0205
USPC 600/440; 600/407

(57) ABSTRACT

A diagnostic imaging apparatus including: an imaging section imaging a site of an examinee to generate image data; a biological signal acquisition section acquiring signal data which is periodical movement of the site of the examinee; a storage section storing the generated image and acquired biological signal data; a biological signal analysis section analyzing the signal data to detect signal waveforms; and a control section calculating evaluation values indicating steadiness of the periodical movement among the respective periods, causing the biological signal analysis section to perform extraction from among the plurality of successive periods on the basis of the calculated evaluation values and a time difference or time ratio threshold range, reading out image data generated during an extracted adaptive period, among the image data stored in the storage section, from the storage section, and causing a display section to display the read-out image data.

### TECHNICAL FIELD

The present invention relates to a diagnostic imaging apparatus and an image display method, and in particular to a technique for displaying an image of a site performing periodical movement such as a heart of an examinee.

### BACKGROUND ART

A doctor examines whether a site performing periodical movement is normal or in a disease condition by observing images of the site performing periodical movement corresponding to a plurality of cycles, which are obtained from a diagnostic imaging apparatus in synchronization with a biological signal which periodically changes, such as an electrocardiographic waveform, pulsation, blood pressure and cardiac sound. Especially, if the site performing periodical movement is a heart, the doctor examines whether the movement of the heart is normal or in a disease condition (cardiac function).

Examples of cardiac function measurement using an ultrasound diagnostic apparatus are disclosed in Patent Literature 1 and Non-Patent Literature 1. In Patent Literature 1 and the like, among ultrasound image data collected in synchronization with heartbeats of successive three cycles, time between the most past heartbeat and the next-cycle heartbeat and time between the next-cycle heartbeat and the heartbeat of the next after the next cycle (the most recent) are measured first as a first heartbeat time and a second heartbeat time, respectively. Next, time difference between the first heartbeat time and the second heartbeat time is calculated. Next, ultrasound image data collected in synchronization with a heartbeat when the calculated time difference is equal to a setting value (threshold range) or below is stored into an image memory.

### CITATION LIST

### Patent Literature

Patent Literature 1: JP-A-01-181852

### Non-Patent Literature

Non-Patent Literature 1: Tomotsugu Tabata, et al., Assessment of LV systolic function in atrial fibrillation using an index of preceding cardiac cycles, Am J Physiol Heart Circ Physiol 281: H573-H580, 2001

### SUMMARY OF INVENTION

### Technical Problem

In Patent Literature 1 and the like, the configuration is such that, when the time difference is beyond the threshold range, the ultrasound image data is not stored into the image memory. Therefore, in order to continually perform collection of ultrasound image data using the ultrasound diagnostic apparatus of Patent Literature 1 and the like, an examiner is required to observe the time difference between the first heartbeat time and the second heartbeat time, which is different according to each examinee, and make adjustment manually so that the time difference is within the threshold range, for each examination. That is, in the prior-art technique in Patent Literature 1 and the like, an unsolved problem as described above is that ultrasound image data is not appropriately displayed for all examinees unless an examiner performs manual adjustment of a threshold range which is different according to each examinee.

Thus, the present invention has been made in view of the above problem, and its object is to provide a diagnostic imaging apparatus and an image display method which are capable of appropriately displaying ultrasound image data for all examinees without an examiner performing manual adjustment of a threshold range.

### Solution to Problem

In order to achieve the above object, the present invention picks up an image of a site performing periodical movement (for example, a heart) of an examinee to generate image data, acquires biological signal data which periodically changes, such as heartbeats of the examinee, detects particular signal waveforms of the acquired biological signal data, stores the generated image data in synchronization with the biological signal data, calculates, on the basis of time difference or time ratio among a plurality of successive periods constituted by intervals among the detected particular signal waveforms, evaluation values indicating steadiness of the periodical movement among the respective periods, performs extraction from among the plurality of successive periods on the basis of the calculated evaluation values and a time difference or time ratio threshold range, reads out image data generated during an adaptive period extracted from the stored image data, and displays the read-out image data.

### Advantageous Effects of Invention

According to the present invention, it is possible to provide a diagnostic imaging apparatus and an image display method which are capable of appropriately displaying ultrasound image data for all examinees without an examiner performing manual adjustment of a threshold range.

### BRIEF DESCRIPTION OF DRAWINGS

FIG. 1 is a block diagram showing the configuration of an ultrasound diagnostic apparatus according to each embodiment.

FIG. 2 is a flowchart showing the flow of a process of a first embodiment.

FIG. 3 is an explanatory diagram showing a process for calculating the evaluation value of an R-R period.

FIG. 4 is an explanatory diagram showing a process for analyzing electrocardiographic data according to the first embodiment; (a) shows processing of data existing at the top of a cine-memory; (b) shows processing executed after (a); and (c) shows processing of data existing at the end of the cine-memory.

FIG. 5 is a schematic diagram showing an example of a display screen according to the first embodiment.

FIG. 6 is an explanatory diagram showing sort processing.

FIG. 7 is a schematic diagram showing a screen display example according to a second embodiment.

FIG. 8 is a schematic diagram showing a screen display example according to the second embodiment.

FIG. 9 is a schematic diagram showing a screen display example according to the second embodiment.

FIG. 10 is a flowchart showing the flow of a process for extracting detection periods and then removing detection periods of tachycardia and bradycardia.

FIG. 11 is an explanatory diagram showing the process for extracting detection periods and then removing detection periods of tachycardia and bradycardia; (a) shows electrocardiographic data; (b) shows a group of detection periods; (c) shows display by a first detection period selection method; (d) shows display by a second detection period selection method; and (e) shows display by a third detection period selection method.

FIG. 12 is a flowchart showing the flow of a process for removing R-R periods corresponding to tachycardia and bradycardia from electrocardiographic data and then detecting detection periods.

FIG. 13 is a flowchart showing the flow of a process of a fourth embodiment.

FIG. 14 is a schematic diagram showing a display example of electrocardiographic data of the fourth embodiment; (a) shows electrocardiographic data immediately after start of analysis; (b) shows an example in which R₁ which will have a better evaluation value has been inputted after (a); (c) shows an example in which R₁ which will have the same evaluation value as the best value 1.20 has been inputted; and (d) shows an example in which R₁ which will have an evaluation value worse than the best value has been inputted.

### DESCRIPTION OF EMBODIMENTS

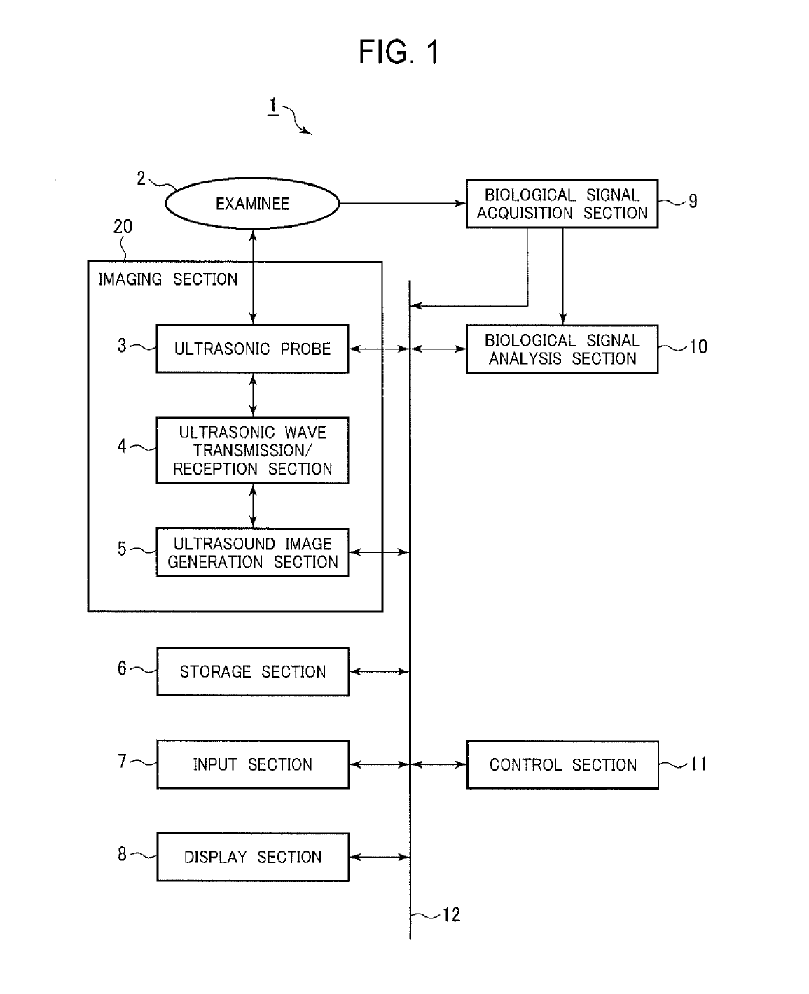

Embodiments of the present invention will be described below with the use of drawings. Components having the same function and procedures having the same processing content are given the same reference numerals, and repetition of description thereof is omitted. In each embodiment described in this document, description will be made with the case of using an ultrasound diagnostic apparatus as a diagnostic imaging apparatus and performing diagnostic imaging of the cardiac function of an examinee. The diagnostic imaging apparatus, however, is not limited to an ultrasound diagnostic apparatus, and an MRI apparatus and an X-ray CT apparatus are also possible. That is, the diagnostic imaging apparatus only has to have an imaging section 20, a storage section 6, an input section 7, a display section 8, a biological signal acquisition section 9, a biological signal analysis section 10 and a control section 11 as shown in FIG. 1.

In the ultrasound diagnostic apparatus, the imaging section 20 is formed with an ultrasound probe 3, an ultrasonic wave transmission/reception section 4, and an ultrasound image generation section 5. Description of detailed functions of each component will be made later. Others. In an MRI apparatus, a static magnetic field generator, a gradient magnetic field generator, a transmission pulse generator and a receiving coil correspond to the imaging section 20. In an X-ray CT apparatus, an X-ray scanner with an X-ray tube and an X-ray detector attached to a rotating disk corresponds to the imaging section 20. Though an electrocardiogram is used as a biological signal resulting from periodical movement of the heart (beats), the pulse, blood pressure, heart sound and the like are also possible.

First, the configuration of an ultrasound diagnostic apparatus according to each embodiment will be described on the basis of FIG. 1. FIG. 1 is a block diagram showing the configuration of the ultrasound diagnostic apparatus according to each embodiment described in this document.

As shown in FIG. 1, an ultrasound diagnostic apparatus 1 is provided with the ultrasound probe 3, the ultrasonic wave transmission/reception section 4, the ultrasound image generation section 5, the storage section 6, the input section 7, the display section 8, the biological signal acquisition section 9, the biological signal analysis section 10, the control section 11 and a system bus 12, and these ultrasound probe 3, ultrasonic wave transmission/reception section 4, ultrasound image generation section 5, storage section 6, input section 7, display section 8, biological signal acquisition section 9, biological signal analysis section 10 and control section 11 are mutually connected by the system bus 12.

The ultrasound probe 3 is configured by arranging transducer elements represented by piezoelectric substances in an array shape. The ultrasound probe 3 is caused to be in contact with an examinee 2 to transmit ultrasonic wave and also to receive a reflected wave reflected in the examinee and generate a reflected echo signal. An ultrasound probe having a scanning method of the linear type, convex type, sector type or the like can be applied to the ultrasound probe 3.

The ultrasonic wave transmission/reception section 4 receives information from the control section 11 about the power and timing of an ultrasonic signal to be transmitted to or received, and it transmits a pulse signal for radiating ultrasonic waves to the ultrasound probe 3 and also performs control for causing the ultrasound probe 3 to acquire a predetermined reflected echo signal. Then, the ultrasonic wave transmission/reception section 4 outputs the reflected echo signal received by the ultrasound probe 3 to the ultrasound image generation section 5.

The ultrasound image generation section 5 causes the reflected echo signal inputted from the ultrasonic wave transmission/reception section 4 to pass into a phasing circuit and an amplification circuit and, furthermore, performs signal processing in accordance with setting of an image given from the control section 11. Then, the ultrasound image generation section 5 generates ultrasound image data, such as, for example, a tomographic image of the biotissue of the examinee 2, a blood flow image and blood flow speed image based on Doppler measurement and a tissue Doppler image, on the basis of the shaped reflected echo signal.

The ultrasound image data generated by the ultrasound image generation section 5 and biological signal data acquired by the biological signal acquisition section 9 are synchronization-processed by the control section 11 and stored into the storage section 6. A program realizing the function of each of the sections constituting the ultrasound diagnostic apparatus 1 is also stored in the storage section 6. For example, an arithmetic algorithm for biological signal analysis executed by the biological signal analysis section 10 is stored.

The input section 7 is an interface used by an examiner who performs various operations of the ultrasound diagnostic apparatus 1 and is provided with input equipment such as, for example, a keyboard, a trackball, switches and a dial. The input section 7 is used, for example, to make biotissue measurement settings on an ultrasound image displayed on a display screen of the display section 8 or to move the current time phase or period of a reproduced image.

The display section 8 displays a biological signal or an ultrasound image on a screen.

The biological signal acquisition section 9 acquires a biological signal of the examinee 2, converts it to biological signal data and stores it into the storage section 6. At the time of causing the biological signal acquisition section 9 to operate in real time, the biological signal acquisition section 9 outputs the biological signal data directly to the biological signal analysis section 10. In each embodiment, an electrocardiograph is configured independently from the ultrasound diagnostic apparatus 1, and the biological signal acquisition section 9 is electrically connected to the electrocardiograph and is configured as an interface of the ultrasound diagnostic apparatus 1 which receives electrocardiographic data from this electrocardiograph. The biological signal acquisition section 9, however, may be configured as an electrocardiograph electrically connected to the ultrasound diagnostic apparatus 1.

The biological signal analysis section 10 detects particular signal waveforms on the basis of the biological signal data read from the storage section 6 or biological signal data inputted from the biological signal acquisition section 9. In each embodiment, R-waves included in an electrocardiographic waveform are detected as the particular signal waveforms. Then, the biological signal analysis section 10 calculates time of an electrocardiographic waveform between adjoining R-waves (hereinafter referred to as an “R-R period” or simply a “period”).

Next, the biological signal analysis section 10 calculates the ratio or time difference between two successive periods preceding an evaluation target period and calculates an evaluation value using this time ratio or time difference. Though the evaluation value is also referred to as an index value using time ratio or time difference here, description will be made below with the use of the term “evaluation value”. Then, the biological signal analysis section 10 judges whether or not the evaluation target period is to be a period to be targeted by examination (hereinafter referred to as a “detection period”) using this evaluation value. The biological signal analysis section 10 also judges whether the R-R period or the detection period corresponds to any of tachycardia and bradycardia. The details of the detection period extraction process and tachycardia/bradycardia judgment process described above will be described later.

The control section 11 is configured being provided with an arithmetic/control device such as a CPU, and it controls the whole of the ultrasound diagnostic apparatus 1. In each embodiment, the control section 11 performs synchronization control of biological signal data and ultrasonic measurement data and performs control for synchronously storing these biological signal data and ultrasonic measurement data into the storage section 6. The control section 11 also controls synchronization of a series of processes related to the display section 8, the biological signal acquisition section 9 and the biological signal analysis section 10.

The system bus 12 is a bus for exchanging data among the respective components.

A measurement arithmetic section which determines measurement data pertaining to the cardiac function, such as a blood flow state, blood flow speed, the speed of an annulus, the capacity of an atrium and movement of the wall of the heart, by arithmetic using a reflected echo signal outputted from the ultrasonic wave transmission/reception section 4 may be further provided though it is not shown in FIG. 1. In this case, the ultrasonic wave transmission/reception section 4 outputs the reflected echo signal to the ultrasound image generation section 5 and the measurement arithmetic section. An arithmetic program of the measurement arithmetic section may be stored in the storage section 6.

### First Embodiment

A first embodiment is an example of so-called an off-line process in which electrocardiographic data to be an analysis target is stored in the storage section 6 in advance and then analysis-processed, and it is an embodiment in which one detection period optimal for diagnostic imaging for each examinee is displayed. That is, biological signal data and ultrasound image data are synchronously stored in the storage section 6, and the biological signal analysis section 10 extracts the detection period on the basis of the biological signal data read from the storage section 6.

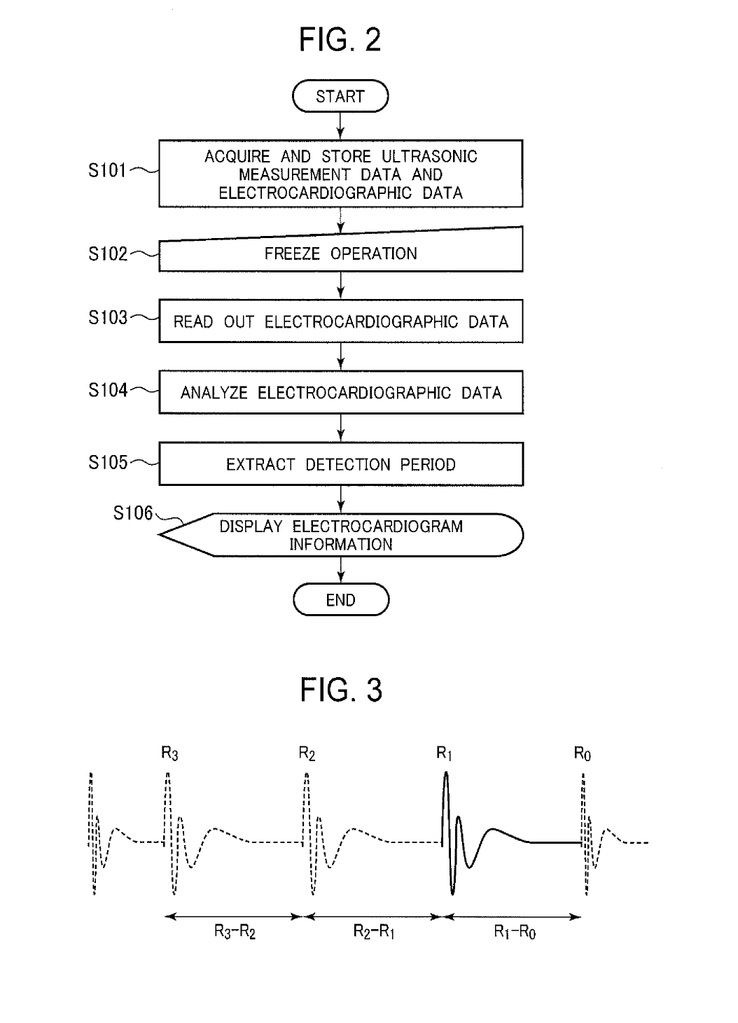

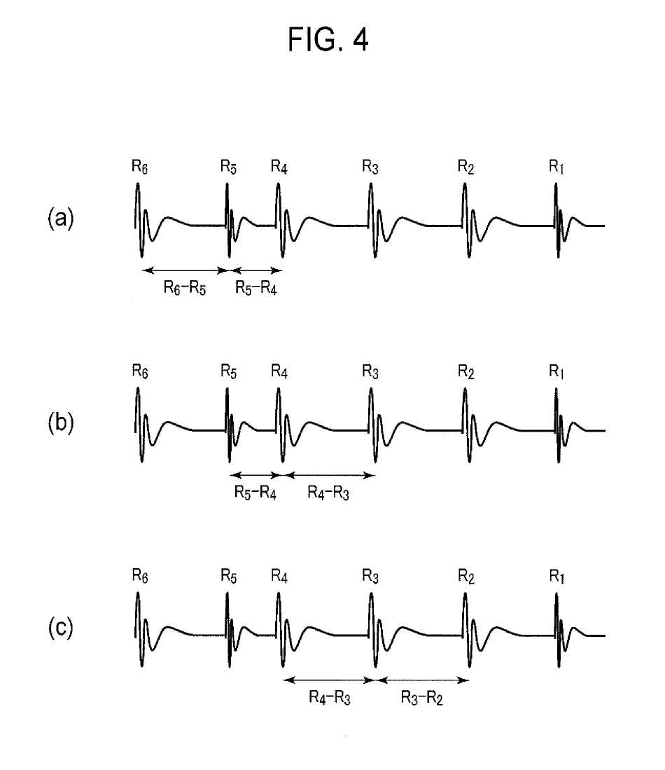

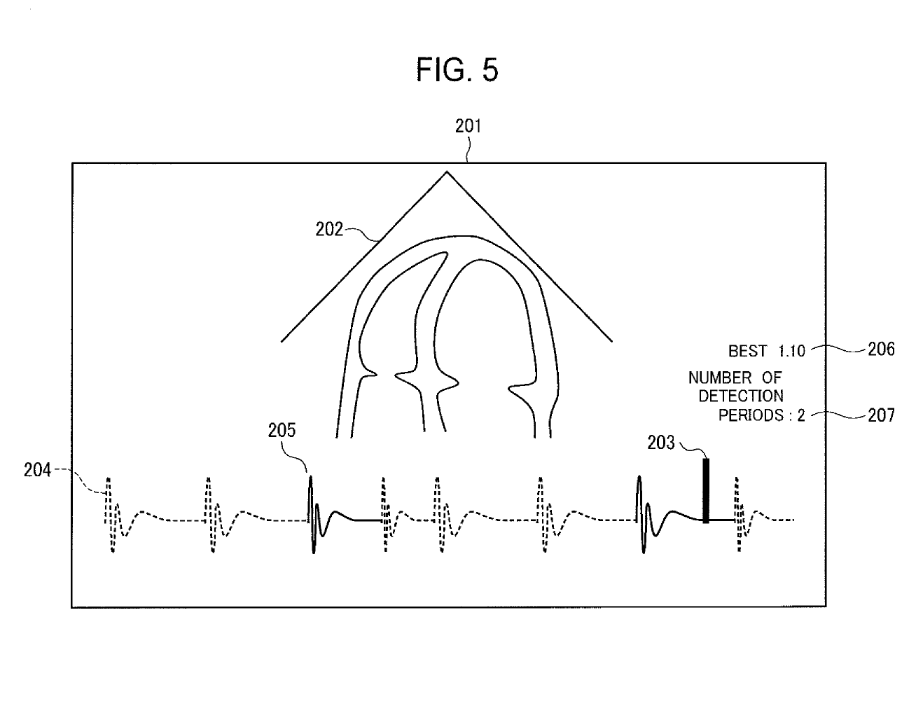

The first embodiment will be described below on the basis of FIGS. 2 to 5. FIG. 2 is a flowchart showing the flow of the process of the first embodiment. FIG. 3 is an explanatory diagram showing a process for calculating the evaluation value of an R-R period. FIG. 4 is an explanatory diagram showing a process for analyzing electrocardiographic data according to the first embodiment; (a) shows processing of data existing at the top of a cine-memory; (b) shows processing executed after (a); and (c) shows processing of data existing at the end of the cine-memory. FIG. 5 is a schematic diagram showing an example of a display screen according to the first embodiment. Description will be made below along the order of respective steps in FIG. 2.

### (Step S101)

The examiner fits an electrocardiograph (the biological signal acquisition section 9) to the examinee 2 to measure electrocardiographic data and causes the ultrasound probe 3 to be in contact with the chest of the examinee 2 to perform ultrasonic measurement by transmitting and receiving ultrasonic waves under predetermined imaging conditions. The ultrasound image generation section 5 generates ultrasonic measurement data which includes ultrasound image data and Doppler measurement data, on the basis of a reflected echo signal which the ultrasonic wave transmission/reception section 4 has outputted. The control section 11 synchronizes the electrocardiographic data and the ultrasonic measurement data with each other and stores them into the storage section 6 (S101). At this time, for example, an electrocardiographic waveform chart and a moving ultrasound image are displayed on the display screen of the display section 8.

### (Step S102)

Next, when the examiner performs a freeze operation from the input section 7, representation on the display screen of the display section 8 is stopped (S102). Since the first embodiment performs cardiac function analysis by an off-line process, the process for synchronizing and storing electrocardiographic data and ultrasonic measurement data by the storage section 6 ends at this step. Then, at and after step S103, cardiac function analysis is performed on the basis of the electrocardiographic data and ultrasonic measurement data stored in the storage section 6.

### (Step S103)

The biological signal analysis section 10 reads out the electrocardiographic data stored in the storage section 6 (S103).

### (Step S104)

The biological signal analysis section 10 starts electrocardiographic data analysis (S104). The R-waves of an electrocardiographic waveform as particular signal waveforms which the biological analysis section 10 detects are set as detection targets. Detection of R-wave is performed by a well-known method such as pattern matching of electrocardiographic waveform.

Next, the biological signal analysis section 10 calculates the evaluation value of each R-R period. In the first embodiment, the ratio of two successive periods adjoining a period to be an evaluation value calculation target is calculated as the evaluation value of each R-R period, and periods are extracted as detection periods in order from an evaluation value closest to 1. The evaluation value calculation process will be specifically described with the use of FIG. 3. In the electrocardiographic data in FIG. 3, for the evaluation value of an R-R period indicated by R₁-R₀ (a period represented by a solid line in FIG. 3), time ratio of two periods R₂-R₁ and R₃-R₂ successively preceding R₁-R₀, that is, (R₂-R₁ period/R₃-R₂ period) is used. If this evaluation value is almost “1”, the time periods of R₂-R₁ and R₃-R₂ are almost the same, and R₁-R₀ is a heartbeat immediately after the two successive steady heartbeats and is evaluated to be an appropriate period for cardiac function measurement.

Therefore, at this step, the biological signal analysis section 10 starts the process at the top of the cine-memory as in FIG. 4(a). The biological signal analysis section 10 detects R-waves from the electrocardiographic data of the cine-memory in order of R₆, R₅ and R₄. When detecting R₅ after detection of R₆, the biological signal analysis section 10 calculates an R₆-R₅ period. Next, when detecting R₄ after detection of R₅, the biological signal analysis section 10 calculates an R₅-R₄ period. Then, when detecting R₃ adjoining R₄, the biological signal analysis section 10 performs calculation of (R₅-R₄ period/R₆-R₅ period) and stores the result as the evaluation value of an R₄-R₃ period. In this case, the evaluation values of the R₆-R₅ and R₅-R₄ periods are not calculated.

Next, as in FIG. 4(b), when R₂ adjoining R₃ is detected, calculation of (R₄-R₃ period/R₅-R₄ period) is performed, and the result is stored as the evaluation value of the R₃-R₂ period. This calculation is repeated, and R₁, which is the last R-wave of the cine-memory, is detected, and calculation up to the evaluation value of R₂-R₁ (R₃-R₂ period/R₄-R₃ period) is performed, as shown in FIG. 4(c).

The R-wave detection and evaluation value calculation/storage described above may be performed in order from the end to top of the cine-memory like in order of (c), (b) and (a) in FIG. 4. The two adjoining heartbeats may be increased to three or four heartbeats. For calculation of an evaluation value using three or more heartbeats, a method of accumulating respective evaluation values calculated with two heartbeats may be used. For example, in the case of calculating an evaluation value using the time ratio of three adjoining heartbeats, the evaluation value may be calculated by calculating evaluation values 1 and 2 using adjoining heartbeats among the three heartbeats and calculating a value of accumulation of the evaluation values as the evaluation value using the three heartbeats by Expression (1) below.

Evaluation value 1=|(preceding R-R)/(further-preceding R-R)−1|

Evaluation value 2=|(further-preceding R-R)/(still-further-preceding R-R)−1|

Evaluation value=evaluation value 1+evaluation value 2 Expression (1)

In the above case, 0 is the optimum evaluation value.

When FIG. 4 is taken up as an example, the following is obtained:

Evaluation value of R₃-R₂ period=|(R₄-R₃ period)/(R₅-R₄ period)−1|+|(R₅-R₄ period)/(R₆-R₅ period)−1|

As another evaluation value, difference between two successive periods adjoining a period to be an evaluation value calculation target may be calculated, and periods may be extracted as detection periods in order from such an evaluation value that the absolute value of the time difference is closest to 0. For example, in the case of calculating an evaluation value using time difference among three adjoining heartbeats, the evaluation value may be calculated by calculating evaluation values 1 and 2 using adjoining heartbeats among the three heartbeats and calculating a value of accumulation of the evaluation values as the evaluation value using the three heartbeats by Expression (2) below.

Evaluation value 1=|(preceding R-R)/(further-preceding R-R)|

Evaluation value 2=|(further-preceding R-R)/(still-further-preceding R-R)|

Evaluation value=evaluation value 1+evaluation value 2 Expression (2)

In the above case also, 0 is the optimum evaluation value.

When FIG. 4 is taken up as an example, the following is obtained:

Evaluation value of R₃-R₂ period=|(R₄-R₃ period)−(R₅-R₄ period)|+|(R₅-R₄ period)−(R₆-R₅ period)|

If two adjoining periods are almost the same when compared, the pulse can be evaluated to be regular and steady. On the other hand, if the difference is large, the pulse can be evaluated to be irregular. Therefore, by analyzing all pairs of adjoining R-R periods, the best evaluation value in the cine-memory is identified.

### (Step S105)

The biological signal analysis section 10 searches for the best evaluation value (best value) among all the evaluation values calculated at step S104 and extracts only a period having the best value as a detection period (S105). The best value defined in this document refers to an evaluation value closest to “1” in the case of using time ratio (R-R ratio) as an evaluation value, and, in the case of using time difference as an evaluation value, for example, in the case of determining the evaluation value of the R₄-R₃ period by {(R₅-R₄ period)−(R₆-R₅ period)} in FIG. 4(a), refers to such an evaluation value that the absolute value of the time difference is the smallest. The biological signal analysis section 10 extracts the best value.

There may be a case where a plurality of periods have the same evaluation value because of the reason of an evaluation value calculation method, rounding off of evaluation values and the like, In such a case, all periods having the best value may be displayed as a detection result, or only the latest period in the cine-memory may be regarded as a detection period. Furthermore, it is also possible to use a plurality of evaluation values of time ratio and time difference as evaluation values and leave only one period which comprehensively has the best value when the plurality of evaluation values are combined.

### (Step S106)

The display section 8 displays a screen showing a detection result (9106).

For example, in FIG. 5, as a screen display example showing a detection result, an electrocardiographic waveform 204 in a range targeted by electrocardiographic data analysis is displayed on a display screen 201. On the electrocardiographic waveform 204, an R-R period 205 detected to have the best value is indicated by a solid line, and other periods are indicated by dotted lines. That is, the display section 8 displays a detection period in a biological signal diagram (corresponding to the electrocardiographic waveform 204) showing biological signal data (corresponding to electrocardiographic data) being identified from the other periods.

The display section 8 displays, on the display screen 201, “BEST 1.10” 206 which is the most favorable evaluation value in the cine-memory (hereinafter referred to as “the best value”) and “number of detection periods: 2” 207 which is the number of periods having the best value, as detailed information. Thereby, if the value of the best value or the number of detection periods displayed is not favorable, it can be judged that a biological signal is to be taken again without confirming the values of the best values or the numbers of detection periods by tracing back the cine-memory, which contributes to procedure improvement.

On the display screen 201, an ultrasound image 202, which is a tomographic image of a heart, is also displayed. This ultrasound image 202 is a tomographic image of a heart at a position on the time axis of a time phase bar 203 displayed on the electrocardiographic waveform 204. That is, the time phase bar 203 shows the time phase on the electrocardiographic waveform of the currently reproduced ultrasound image 202. The time phase bar 203 may be positioned on the detection period 205 in the initial state of the display screen 201. Thereby, an ultrasound image of a detection period showing the best value can be initially displayed. This display control of the ultrasound image 202 is realized by the display section 8 reading out an ultrasound image at the time phase of the time phase bar 203 from the storage section 6 and displaying the ultrasound image.

As described above, since the diagnostic imaging apparatus according to the first embodiment is provided with the imaging section 20 picking up an image of a site of the examinee 2 to generate image data; the biological signal acquisition section 9 acquiring biological signal data which is periodical movement of the site of the examinee 2; the storage section 6 synchronously storing the generated image data and the acquired biological signal data; the biological signal analysis section 10 analyzing the biological signal data to detect particular signal waveforms; and the control section 11 calculating, on the basis of time difference or time ratio among a plurality of successive periods constituted by intervals among the detected particular signal waveforms, evaluation values indicating steadiness of the periodical movement among the respective periods, performing extraction from among the plurality of successive periods on the basis of the calculated evaluation values and a time difference or time ratio threshold range, and reading out image data generated during an extracted adaptive period, among the image data stored in the storage section 6, from the storage section 6; and the display section 8 displaying the read-out image data, it is possible to appropriately display ultrasound image data for all examinees without an examiner performing manual adjustment of a threshold range.

In the first embodiment, the biological signal analysis section extracts a period having the best evaluation value as a detection period. Thereby, the examiner can quickly find the best value and the detection period he is most interested in. Furthermore, since the best value is calculated on the basis of a biological signal of each examinee and is not extracted by comparison with a particular threshold range, it is possible to calculate and display the best value of each examinee irrespective of the condition of the examinee and avoid trouble in which the best value is not displayed at all.

### Second Embodiment

The second embodiment is an example of the so-called off-line process similarly to the first embodiment. However, while the first embodiment is an embodiment in which only a period having the best value is displayed as a detection period, the second embodiment is characterized in that the number of detection periods to be left as a detection result is specified. The merits of the second embodiment are a point that, for example, in the case where, even though a best-value period is detected, an ultrasound image stored in the storage section 6 cannot be used for cardiac function measurement because of being disturbed by influence of an irregular pulse, the cardiac function measurement can be performed with the ultrasound image of another detection period, and a point that, from a viewpoint that it is generally recommended that measurement results with a plurality of numbers of heartbeats should be averaged in the case of an irregular pulse, the convenience of cardiac function measurement for an examinee with an irregular pulse is improved.

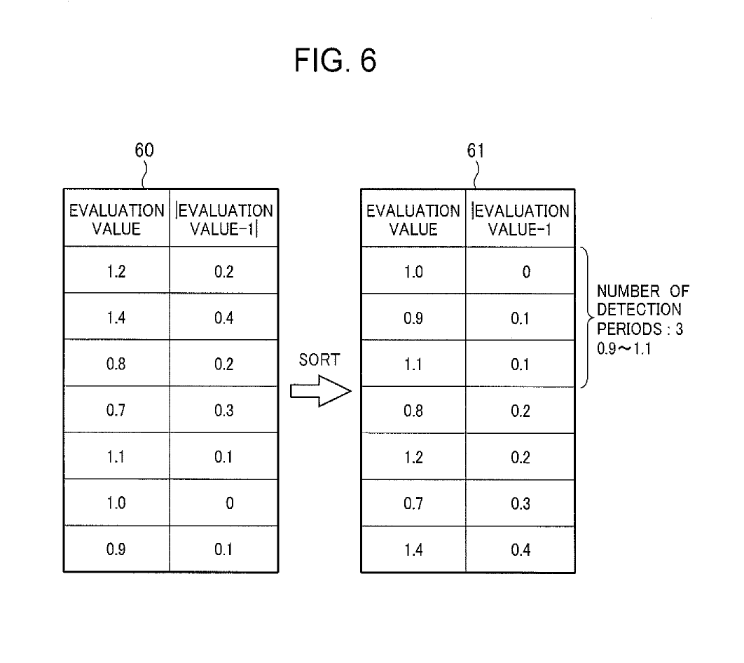

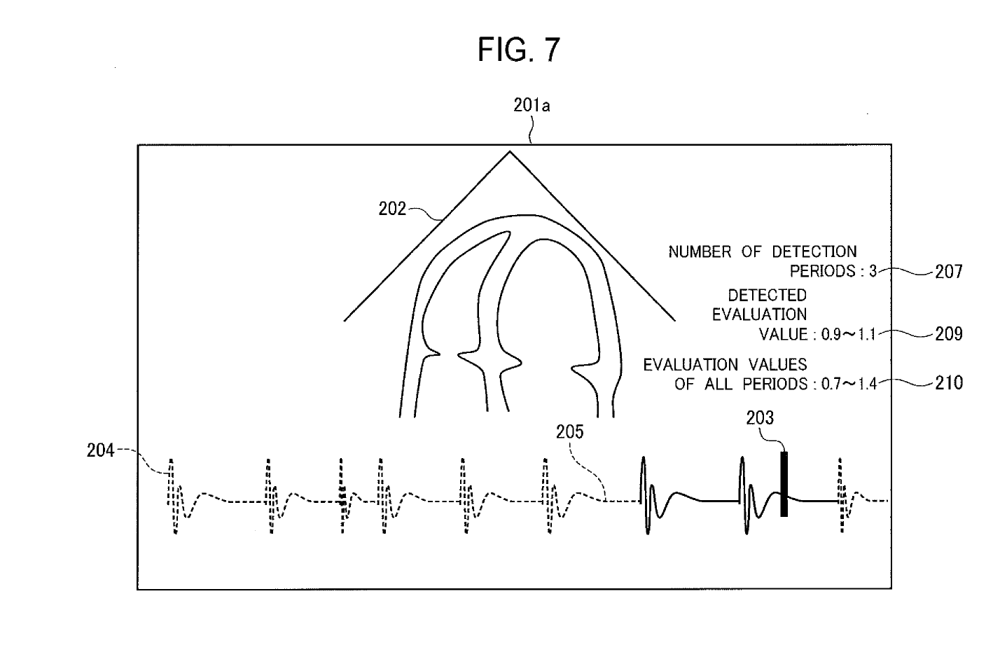

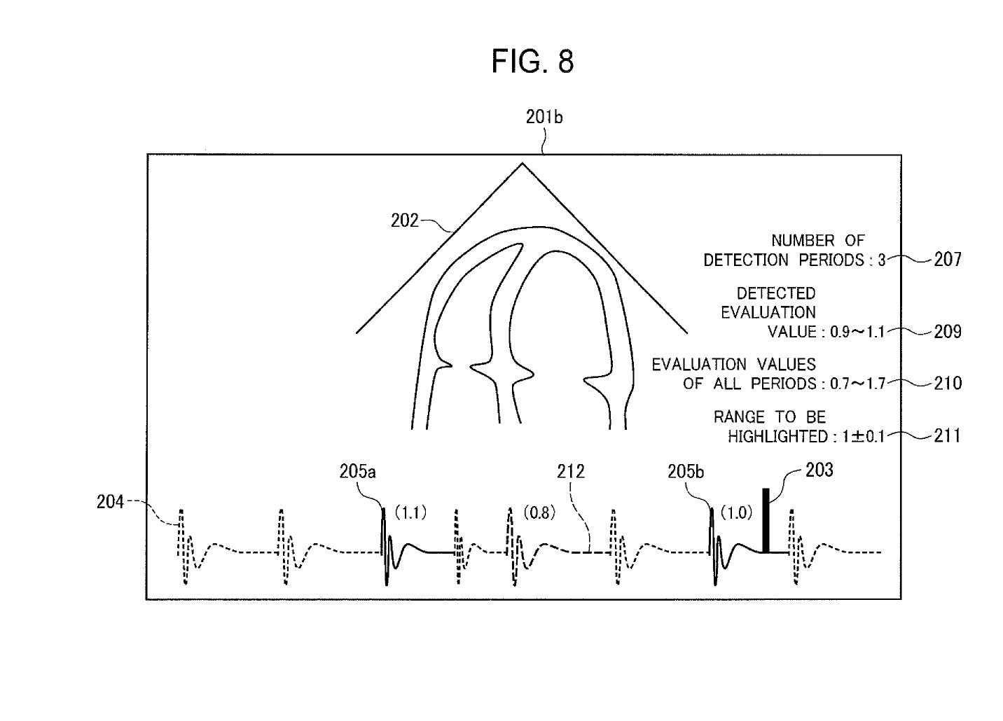

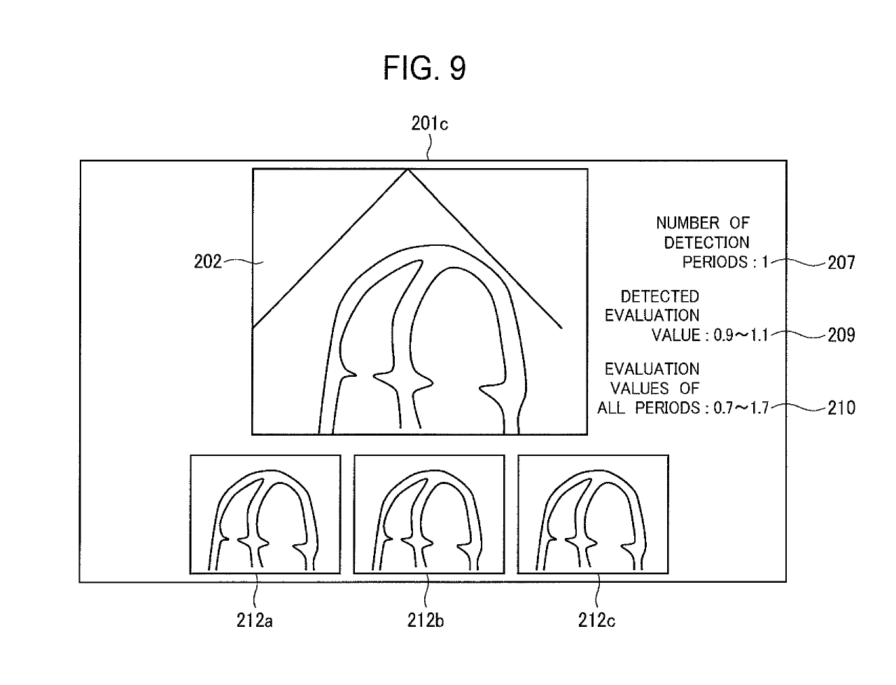

The second embodiment will be described below on the basis of FIGS. 6 to 9. FIG. 6 is an explanatory diagram showing sort processing. FIG. 7 is a schematic diagram showing a screen display example according to the second embodiment. FIG. 8 is a schematic diagram showing a screen display example according to the second embodiment. FIG. 9 is a schematic diagram showing a screen display example according to the second embodiment. Since the flow of the process of the second embodiment is similar to the flow of the process of the first embodiment, FIG. 2 will be applied, and description will be made only on different points. Description will be made below along the order of steps in FIG. 2.

From step S101 to step S104 in FIG. 2, a process similar to that of the first embodiment is performed. When step S104 ends, evaluation values of all periods are calculated and stored from electrocardiographic data existing in the cine-memory. Here, the biological signal analysis section 10 calculates a second evaluation value for sort processing to be executed at the next step. The biological signal analysis section 10 calculates the second evaluation value which indicates deviation of the evaluation value of each period relative to an evaluation value which appears when the periodical movement is steady (hereinafter referred to as a “desirable evaluation value”). For example, since the “desirable evaluation value” is “1” when the evaluation value is the R-R ratio, the absolute value of (each evaluation value−1) is determined as the second evaluation value, and each evaluation value and a second evaluation value corresponding to the evaluation value are calculated and stored in association with each other. The “desirable evaluation value” described above is “1” when time ratio (R-R ratio) is used as an evaluation value and is “0” when time difference is used as an evaluation value.

Thus, at step S105, the biological signal analysis section 10 performs sort processing in ascending order of the second evaluation values of the respective periods, and extracts detection periods corresponding to the number of detection periods specified, in order from the highest position as a result of the sort processing (S105). Then, the display section 8 displays a detection result (S106).

In the second embodiment, a first specification section which specifies the number of periods to be extracted as detection periods is provided. The first specification section is configured so that, for example, an examiner presets the number of detection periods on a screen of the display section 8 in advance or specifies change in the number of detection periods by operating the input section 7 after displaying a detection result once. If the number of detection periods is reflected on the display screen immediately when an operation of specifying the number of detection periods is performed, the convenience is further enhanced. When the number of detection periods is set to 1, it is useful for the sort processing for detecting the best value in the first embodiment. The sort processing may be performed in descending order of second evaluation values, and detection periods corresponding to the specified number of detection periods are extracted in order from the lowest position as a result of the sort processing.

FIG. 6 shows a specific example of the sort processing. It is assumed that, as a result of the analysis of step S104, evaluation values using R-R ratios and second evaluation values corresponding to the respective evaluation values (|evaluation value−1|) are calculated as in Table 60 in FIG. 6. The biological signal analysis section 10 performs sort processing for the second evaluation values in descending order with the best first. Table 61 shows a sorting result. If the specified number of detection periods is, for example, 3, the highest three values of “|evaluation value−1|” in Table 61 are caused to be a detection result. As a result, the range of the evaluation values of the detection periods can be calculated as 0.9 to 1.1 from the lower limit value and the upper limit value.

FIG. 7 shows one of screen display examples. In this example, the “number of detection periods” is set to 3. On a display screen 201a in FIG. 7, a “number of detection periods” field 208 and a “detected evaluation value” field 209, which shows the range of detected evaluation values, are displayed. An “evaluation values of all periods” field 210 showing the range of the evaluation values of all the R-R periods calculated at step S104 is also displayed. As for display content similar to that of the display screen 201 on the display screen 201a, the same reference numerals will be given, and repeated description thereof will be omitted.

In the case of an examinee without an irregular pulse, the range of evaluation values of detection periods (“detected evaluation value” 209) and the range of evaluation values of all R-R periods (“evaluation values of all periods” 210) concentrate near 1.0 (for example, a result of 0.99 to 1.01 or the like is obtained). As the degree of serious condition of irregular pulse increases, the range gradually spreads. Therefore, there is a merit that, by displaying the range of evaluation values of detection periods (the “detected evaluation value” 209) and the range of evaluation values of all R-R periods (the “evaluation values of all periods” 210), the degree of serious condition of the irregular pulse of an examinee can be judged at a glance. Furthermore, the examiner can judge whether it is necessary or not to take electrocardiographic data again by referring to these values.

Next, another screen display example of the second embodiment will be described on the basis of FIG. 8. In this example, it is assumed that the examiner can set the number of detection periods and the range of evaluation values to be highlighted. These settings can be made by inputting and setting numerical values of the “number of detection periods” field 208 and a “range to be highlighted” field 211 showing the range of evaluation values to be highlighted, on the screen in FIG. 8. That is, in this example, the ultrasound diagnostic apparatus is further provided with a second specification section which specifies the range of evaluation values to be highlighted, and the display section 8 highlights a period showing an evaluation value included in the range of evaluation values to be highlighted, in a biological signal diagram (corresponding to the electrocardiographic waveform 204) showing biological signal data (corresponding to the electrocardiographic data described above).

In FIG. 8, when the number of detection periods is set to “3” in the “number of detection periods” field 208, the highest three values among evaluation values are displayed. It can be set that, when “1±0.1” is inputted in the “range to be highlighted” field 211, detection periods the evaluation values of which are included in 0.9 to 1.1 are highlighted. Therefore, three detection periods 205a, 205b and 212 are displayed being distinguished from other periods in the electrocardiographic waveform 204, and the detection periods 205a (evaluation value 1.1) and 205b (evaluation value 1.0) belonging to an evaluation value range to be highlighted, among the three detection periods, are especially highlighted (represented by solid lines in the electrocardiographic waveform 204), while the detection period 212 (evaluation value 0.8) is not highlighted (represented by a relatively rough broken line in the electrocardiographic waveform 204). Highlighting may be performed by changing the color or thickness of the electrocardiographic waveform 204 or overlapping an object on a relevant period.

Furthermore, in a display screen 201b in FIG. 8, the “detected evaluation value range” field 209 which indicates the range of detected evaluation values and the “evaluation values of all periods” field 210 which indicates the range of evaluation values of all R-R periods are also displayed.

According to the image display example in FIG. 8, it is possible to, while leaving the merit that a plurality of detection periods from the best value for an examinee can be displayed, further specify the range of evaluation values which the examiner wants to distinguishingly display.

By this highlighting, it becomes possible for the examiner to make utilization such as performing cardiac function measurement with the detection results 205a and 205b within the evaluation value range he has specified, first, and, as for the period 212 which is detected but is outside the specified range, only referring to it, which further enhances usability. Though the examiner expresses the range of highlighting in the form of 1±x (x is an arbitrary numerical value) in the example in FIG. 8, it is also possible to set an upper limit value and a lower limit value.

Next, another screen display example of the second embodiment will be described on the basis of FIG. 9. In this example, the number of detection periods is set; ultrasound images of detection periods are displayed being arranged in relatively small screens; and an ultrasound image of a detection period having the best value is enlargedly displayed.

In FIG. 9, the number of detection periods is set to “3” in the “number of detection periods” field 208. The biological signal analysis section 10 extracts detection periods having the highest three evaluation values in electrocardiographic data. The display section 8 reads out ultrasound images corresponding to the detection periods from the storage section 6 and arranges and displays the ultrasound images at a lower part of a display screen 201c in FIG. 8. Ultrasound images 212a, 212b and 212c are ultrasound images of the respective detection periods displayed with the use of the small screens. The display section 8 also displays an enlarged ultrasound image 202 obtained by enlarging the ultrasound image 212a of the best-value detection period among the detection periods. The ultrasound images 212a and 202 of the best-value detection period may be displayed being distinguished from the other ultrasound images 212b and 212c (represented with thick frames in FIG. 9).

When the examiner drags and drops the small-screen ultrasound image 212b or 212c into a display area of the enlarged ultrasound image 202 using the input section 6, the dropped ultrasound image in the small screen is updatedly displayed on the enlarged ultrasound image 202.

As described above, the diagnostic imaging apparatus according to the second embodiment has an advantage specific to the second embodiment in addition to the advantage of the first embodiment.

That is, in the example in FIG. 9, since the display section 8 displays images based on image data (ultrasound images) acquired during detection periods in relatively small display areas (corresponding to the ultrasound images 212a, 212b and 212c) and enlargedly displays an image based on image data of a detection period having the best evaluation value (corresponding to the enlarged ultrasound image 202) in a relatively large display area, it is possible to, while leaving the merit that a plurality of detection periods from the best value in an examinee can be displayed, further confirm ultrasound images of the detection periods. Therefore, it becomes easier to proceed to cardiac function measurement work using the ultrasound images.

The specification of an evaluation value range to be highlighted in FIG. 8 may be combined with the example in FIG. 9. In this case, an ultrasound image within the evaluation value range to be highlighted may be highlighted instead of highlighting of the best value.

### Third Embodiment

A third embodiment is an embodiment in which tachycardia and bradycardia which are generally not used for cardiac function measurement are removed to extract detection periods. The third embodiment may be used together with the first and second embodiments.

Prior to the process, an R-R period (corresponding to a period of time (seconds) of one heartbeat) to be judged as tachycardia and an R-R period to be judged as bradycardia are preset for the biological signal analysis section 10.

The values may be set by an examiner. The tachycardia and bradycardia may be specified by the number of heartbeats in addition to the R-R period described above.

In the case of using the number of heartbeats for judging tachycardia and bradycardia, the number of heartbeats is converted to time per heartbeat for use. For example, if the case where the number of heartbeats per minute is 100 or larger is defined to be tachycardia, the number of heartbeats is converted to R-R time (one period) by calculation of Time per heartbeat: 60 (seconds)/100 (times)=0.6 (seconds), and a period with an R-R time of 0.6 (seconds) or fewer is judged to be tachycardia. If the case where the number of heartbeats per minute is 40 or smaller is defined to be bradycardia, the number of heartbeats is converted to R-R time by calculation of Time per heartbeat: 60 (seconds)/40 (times)=1.5 (seconds), and a period with an R-R time of 1.5 (seconds) or more is judged to be bradycardia.

In the third embodiment, there are two aspects: (1) an aspect of extracting detection periods and then removing detection periods of tachycardia and bradycardia, and (2) an aspect of removing tachycardia and bradycardia from electrocardiographic data and then extracting detection periods. The respective aspects will be described below in order.

(1) Aspect of Extracting Detection Periods and then Removing Detection Periods of Tachycardia and Bradycardia

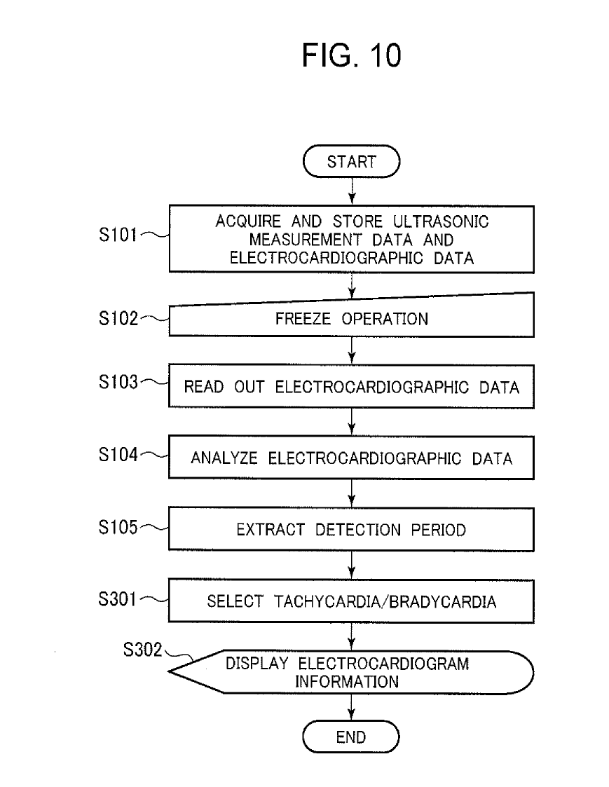

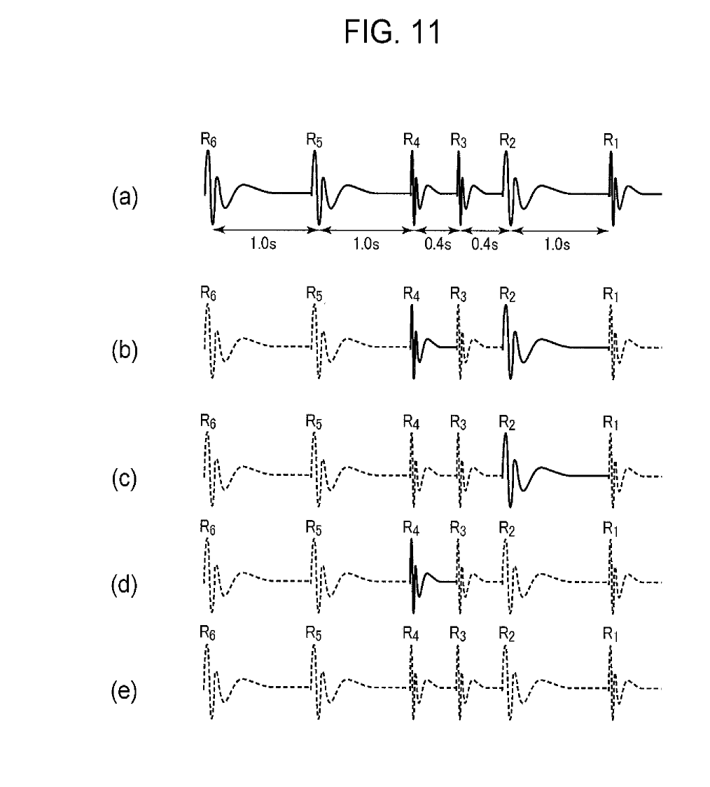

The aspect of (1) will be described on the basis of FIGS. 10 and 11. FIG. 10 is a flowchart showing the flow of a process for extracting detection periods and then removing detection periods of tachycardia and bradycardia. FIG. 11 is an explanatory diagram showing the process for extracting detection periods and then removing detection periods of tachycardia and bradycardia; (a) shows electrocardiographic data; (b) shows a group of detection periods; (c) shows display by a first detection period selection method; (d) shows display by a second detection period selection method; and (e) shows display by a third detection period selection method.

In FIG. 10, steps S101 to S105 are the same as steps S101 to S105 of the first embodiment, and, therefore, description thereof will be omitted.

### (Steps S301 and S302)

The biological signal analysis section 10 selects detection periods corresponding to tachycardia and bradycardia from the detection periods detected at step S105. Then, by removing the selected detection periods, the biological signal analysis section 10 leaves only detection periods included in a range which is neither tachycardia nor bradycardia (S301). Then, the display section 8 displays only the remaining detection periods (S302).

A tachycardia/bradycardia judgment process will be described on the basis of FIG. 11. It is assumed that electrocardiographic data of FIG. 11(a) is inputted. The numerical value below of each R-R period indicates each period. It is assumed that R₄-R₃ and R₂-R₁ are detected as detection periods as shown in FIG. 11(b). (Solid-line periods in FIG. 11(b) are the detection periods.) As methods for selecting detection periods in consideration of tachycardia and bradycardia, for R₄-R₃ and R₂-R₁, which are the detection periods in FIG. 11(b), the following three methods exist.

(1-1) First Detection Period Selection Method: A Method of Comparing Detection Periods with R-R Periods of Tachycardia and Bradycardia

The biological signal analysis section 10 compares a detection period with R-R periods at the time of tachycardia and bradycardia which are set in advance. Then, if the detection period is the R-R time specified as be tachycardia, 0.6 (seconds) or fewer in the above example, the biological signal analysis section 10 judges the detection period to be tachycardia. If the detection period is the R-R time specified as bradycardia, 1.5 (seconds) or more in the above example, the biological signal analysis section 10 judges the detection period to be bradycardia. Then, the biological signal analysis section 10 excludes the detection period judged to be tachycardia or bradycardia. In FIG. 11, since R₄-R₃ is 0.4 seconds, R₄-R₃ is judged to be tachycardia and removed (since being equal to or below a first set time of 0.6, R₄-R₃ is judged to be “tachycardia” and removed).

As a result, at step S302, only R₂-R₁ is displayed as a detection period as shown in FIG. 11(c). According to the method of (1-1) described above, since the biological signal analysis section 10 compares detection periods with predetermined first set time (0.6 seconds which is the R-R time at the time of tachycardia) and with second set time (1.5 seconds which is the R-R time at the time of bradycardia) and extracts a detection period longer than the first set time and shorter than the second set time, trouble does not occur in which a detection period corresponds to tachycardia or bradycardia.

(1-2) Second Detection Period Selection Method: A Method of Comparing R-R Periods Used for Calculation of the Evaluation Values of Detection Periods with R-R Periods of Tachycardia and Bradycardia

The biological signal analysis section 10 judges whether R-R periods used for calculation of the evaluation values of a detection period do not correspond to any of tachycardia and bradycardia, and, if any one of them corresponds to tachycardia or bradycardia, removes the detection period. Thereby, it is possible to remove a detection period detected as a result of performing biological analysis using R-R periods in which tachycardia and bradycardia are included. In FIG. 11(b), R₂-R₁ is detected as a detection period because the evaluation value using the ratio of R₃-R₂ and R₄-R₃ is favorable. However, since both of R₃-R₂ and R₄-R₃ are 0.4 s, the biological signal analysis section 10 judges R₃-R₂ and R₄-R₃ to be tachycardia. Then, the biological signal analysis section 10 excludes R₂-R₁ for which the evaluation value has been calculated from R₃-R₂ and R₄-R₃ from detection periods. As a result, in the display processing at step S302 in FIG. 10, only R₄-R₃ is displayed as a detection result as shown in FIG. 11(d). According to the method of (1-2) described above, since the biological signal analysis section 10 compares periods used for calculation of the evaluation values of detection periods and the predetermined first set time and second set time and extracts a detection period for which the evaluation value has been calculated with the use of only periods longer than the first set time and shorter than the second set time, trouble does not occur in which a detection period influenced by tachycardia or bradycardia is extracted.

### (1-3) Third Detection Period Selection Method: A Method of Combining the First and Second Detection Period Selection Methods

The biological signal analysis section 10 judges whether tachycardia or bradycardia is included in any of a detection period and an R-R period used for the calculation of the evaluation value of the detection period, and, if tachycardia or bradycardia is included in any of them, removes the detection period.

For example, in the case of FIG. 11(b), though R₂-R₁ and R₄-R₃ are detected as detection periods, R₄-R₃ is removed because it is 0.4 seconds and is tachycardia. Furthermore, R₂-R₁ is removed because R₃-R₂ and R₄-R₃ used for biological signal analysis of R₂-R₁ are tachycardia. As a result, in the display processing at step S302 in FIG. 10, it is displayed that there is not a detection period, as shown in FIG. 11(e).

(2) Aspect of Removing Tachycardia and Bradycardia from Electrocardiographic Data and then Extracting Detection Periods

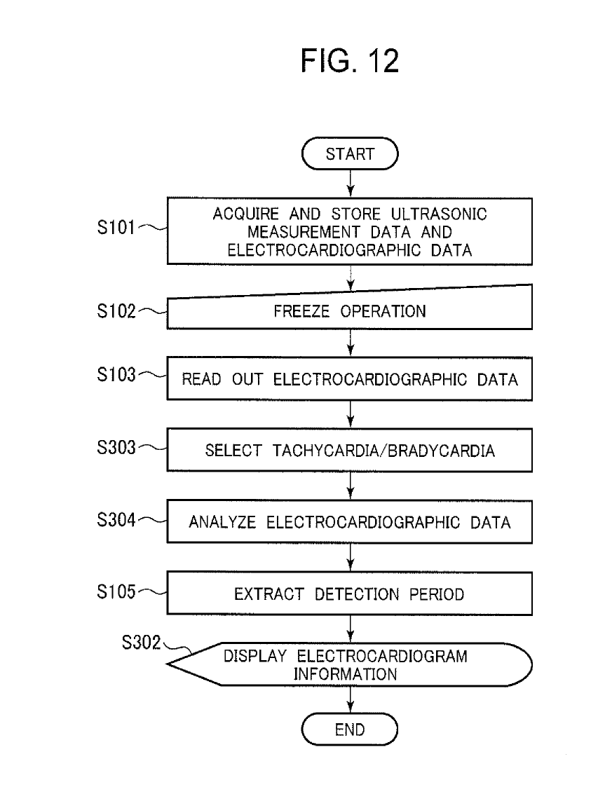

The aspect of (2) will be described on the basis of FIG. 12. FIG. 12 is a flowchart showing the flow of a process for removing R-R periods corresponding to tachycardia and bradycardia from electrocardiographic data and then detecting detection periods.

In FIG. 12, steps S101 to S103 are the same as steps S101 to S103 of the first embodiment, and, therefore, description thereof will be omitted.

### (Step S303)

The biological signal analysis section 10 extracts R-waves, which are particular waveform signals, from electrocardiographic data. Then, the biological signal analysis section 10 judges, for all the R-R periods of the electrocardiographic data, whether they do not correspond to tachycardia or bradycardia, and removes a period corresponding to tachycardia or bradycardia (S303).

For example, in the above example, an R-R period with 0.6 seconds or fewer, or 1.5 seconds or more is removed (is not targeted by the next electrocardiographic data analysis).

### (Step S304)

The biological signal analysis section 10 calculates the evaluation value of each of R-R periods remaining after the period corresponding to tachycardia or bradycardia is removed at step S303 (S304). Since the R-R period of tachycardia or bradycardia has been removed from the electrocardiographic data, the biological signal analysis section 10 determines the evaluation value of the last R-R period among successive three R-R periods using the ratio of preceding two R-R periods among the successive three R-R periods, among the remaining R-R periods.

### (Steps S105 and S302)

The biological signal analysis section 10 detects a detection period on the basis of the evaluation values (S105) similarly to S105 in the first embodiment, and displays a result of the detection (S302) similarly to step S302 in FIG. 10.

According to the aspect of (2), by comparing all periods of biological signal data (corresponding to the electrocardiographic data described above) with predetermined first set time and second set time, extracting only periods with time longer than the first set time and shorter than the second set time, and, on the basis of a period constituted by at least three or more successive such periods, extracting a detection period, periods of tachycardia and bradycardia are removed before analysis of electrocardiographic data. Therefore, the necessity of evaluation value calculation using tachycardia and bradycardia is eliminated, which leads to reduction of processing.

As described above, the diagnostic imaging apparatus according to the third embodiment has an advantage specific to the third embodiment in addition to the advantage of the first embodiment.

Though description has been made with evaluation value calculation using time ratio or time difference as an example in the third embodiment, the present invention can be also applied in the case of extracting detection periods using another evaluation value calculation algorithm. For example, shape matching of successive electrocardiographic waveforms may be used as the evaluation value calculation algorithm. In this case, the shape matching of electrocardiographic waveforms tends to require longer processing time in comparison with the method of calculating an evaluation value using time difference or time ratio. Therefore, by removing periods of tachycardia and bradycardia and then extracting detection periods like the third embodiment, periods targeted by calculation are reduced, and it is more effective against reduction (speed-up) of processing time.

### Fourth Embodiment

A fourth embodiment is an embodiment in which analysis processing is performed at the same time when electrocardiographic data targeted by analysis is successively stored into the storage section 6. That is, the fourth embodiment is an example of a so-called real-time process. More specifically, in the fourth embodiment, the biological signal analysis section 10 acquires biological signal data from the biological signal acquisition section 9 in real time to extract detection periods, and the display section 8 performs updated display of an image picked up during a new detection period.

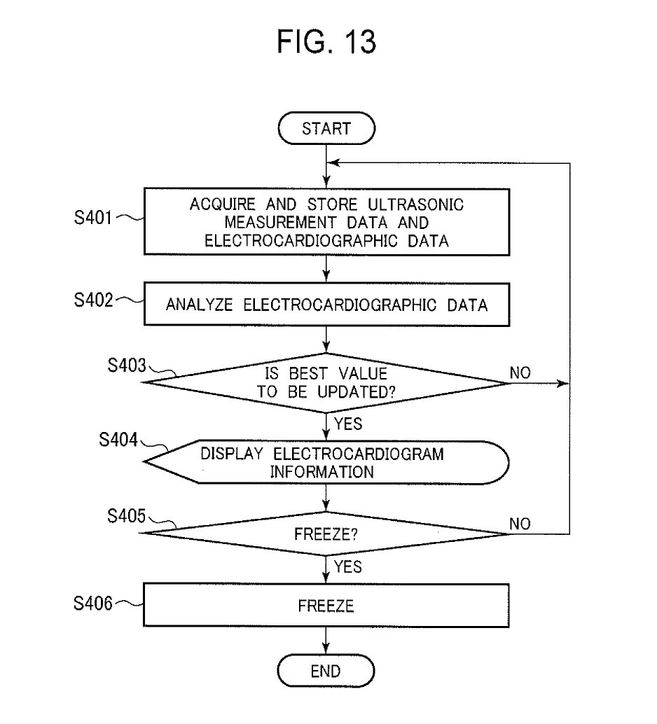

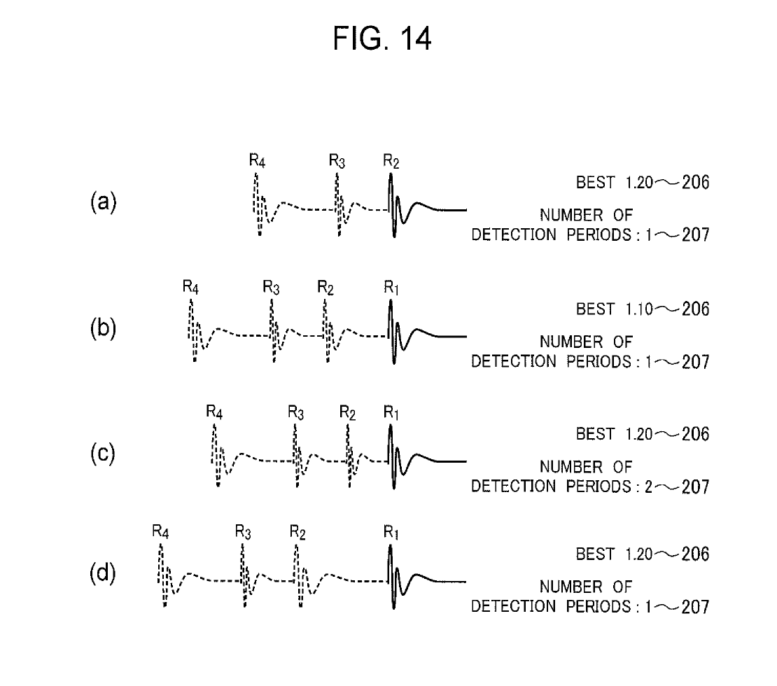

The fourth embodiment will be described below on the basis of FIGS. 13 and 14. FIG. 13 is a flowchart showing the flow of a process of the fourth embodiment. FIG. 14 is a schematic diagram showing a display example of electrocardiographic data of the fourth embodiment; (a) shows electrocardiographic data immediately after start of analysis; (b) shows an example in which R₁ which will have a better evaluation value has been inputted after (a); (c) shows an example in which R₁ which will have the same evaluation value as the best value 1.20 has been inputted; and (d) shows an example in which R₁ which will have an evaluation value worse than the best value has been inputted. Description will be made below along the order of respective steps in FIG. 13.

### (Step S401)

An examiner fits an electrocardiograph to an examinee 2 to measure electrocardiographic data and causes the ultrasound probe 3 to be in contact with the chest of the examinee 2 to perform ultrasonic measurement by transmitting and receiving ultrasonic waves under predetermined imaging conditions. The ultrasound image generation section 5 generates ultrasonic measurement data which includes ultrasound image data and Doppler measurement data, on the basis of a reflected echo signal which the ultrasonic wave transmission/reception section 4 has received. The storage section 6 synchronously stores the electrocardiographic data and the ultrasonic measurement data (S401). An electrocardiographic waveform chart and a moving ultrasound image are displayed on the display screen of the display section 8 (S401).

### (Step S402)

The biological signal analysis section 10 performs analysis of the electrocardiographic data successively inputted (S402). That is, the biological signal analysis section 10 calculates an evaluation value using the latest two R-R periods stored in the storage section 6 (S402).

### (Step S403)

The biological signal analysis section 10 judges whether or not the best value is to be updated with the evaluation value calculated at step S402 (S403). If “Yes”, the biological signal analysis section 10 proceeds to step S404. If “No”, the biological signal analysis section 10 returns to step S401 and continues acquisition of electrocardiographic data. Since the evaluation value determined first becomes the best value, the biological signal analysis section 10 proceeds to “Yes” in the initial loop from S401 to S403. From the next loop, the biological signal analysis section 10 compares the best value stored and an evaluation value determined at the immediately previous step S402.

### (Step S404)

Similarly to the first embodiment, the display section 8 displays information about electrocardiographic data such as the best value 206 and the detected number of periods 207 on the display screen 201 as in FIG. 5 (S404).

### (Step S405)

It is judged whether a freeze command from the input section 7 exists or not. If “Yes”, the biological signal analysis section 10 proceeds to step S406. If “No”, the biological signal analysis section 10 returns to step S401 and continues acquisition of electrocardiographic data.

### (Step S406)

Storage of new electrocardiographic data into the storage section 6 is stopped, and the process of the biological signal analysis section 10 ends (S406).

Display during the process of the fourth embodiment will be described on the basis of FIG. 14. FIG. 14(a) shows electrocardiographic data immediately after start of analysis. Since an evaluation value calculated from comparison between the first two heartbeats R₄-R₃ and R₃-R₂ at the time of starting the analysis is the best value at that time point, an evaluation value constituted by the ratio of R₃-R₂ and R₄-R₃ is displayed in the best value display field 206 as “BEST: 1.20” as in FIG. 14(a), The number of detection periods having the same evaluation value in electrocardiographic data from the start of the analysis is displayed in the “number of detection periods” field 207. Since FIG. 14(a) shows the electrocardiographic data immediately after the analysis, the number is “1”.

FIG. 14(b) is an example in which R₁, which will have a better evaluation value, is inputted after FIG. 14(a). If an evaluation value calculated from comparison between the next R₂-R₁ and R₃-R₂ is 1.10, a result still better than 1.20 which is the best value at that time point, the displayed best value is updated with the new evaluation value. Therefore, “BEST: 1.10” is displayed in the best value display field 206. In the “number of detection periods” field 207, “1”, which is the number of detection periods having the best value 1.10, is displayed.

FIG. 14(c) is an example in which R₁, which will have the same evaluation value as the best value 1.20, is inputted after FIG. 14(a). If an evaluation value calculated from comparison between the next R₂-R₁ and R₃-R₂ is the same value as 1.20 which is the best value at that time point, the best value 1.20 is not updated, and the “number of detection periods” field 207 is updatedly displayed with “2”.

FIG. 14(d) is an example in which R₁, which will have an evaluation value worse than the best value, is inputted after FIG. 14(a). If an evaluation value calculated from comparison between the next R₂-R₁ and R₃-R₂ is worse than 1.20 which is the best value at that time point, neither the best value nor the number of detection periods is updated.

As described above, the diagnostic imaging apparatus according to the fourth embodiment has an advantage specific to the fourth embodiment in addition to the advantage of the first embodiment.

According to the fourth embodiment, since it is possible to confirm the best value stored in the cine-memory (storage section 6) in real time, it becomes easier to determine the timing of stopping storage of electrocardiographic data.

When an evaluation value is displayed being attached to each R-R period, the evaluation value immediately goes out of the screen and disappears because, in real time, the waveform of an electrocardiogram constantly flows in a horizontal direction of the screen. Even if an evaluation value is displayed being fixed at one position on the screen, it is difficult to follow the evaluation value with eyes because a new heartbeat is inputted in less than one second and the evaluation value is updated. However, by updating only information about the best value as appropriate like the fourth embodiment, information to which the examiner pays attention is limited, which leads improvement of usability.

By making it possible to set an evaluation value threshold range and the number of times of the evaluation value exceeding the threshold range in advance, and providing a mechanism for automatically performing freeze at the time point when the evaluation value exceeding the threshold range is detected the set number of times or more, usability is improved more.

Though description has been made above with the update of the “best value” and the number of periods corresponding to the “best value” in real time as an example, a range of evaluation values corresponding to the “number of detection periods” may be updatedly displayed in real time. That is, at step S403 in FIG. 13, evaluation values which have been detected by that time point are continually sorted in order with the best first, and a range of evaluation values corresponding to the specified number of periods from the best value, for example, the range of the highest three evaluation values is displayed. In this case, a range of evaluation values, for example “0.9 to 1.1” is displayed instead of the best value 206 in FIG. 14.

### REFERENCE SIGNS LIST

1 ultrasound diagnostic apparatus
2 examinee
3 ultrasound probe
4 ultrasonic wave transmission/reception section
5 ultrasound image generation section
6 storage section
7 input section
8 display section
9 biological signal acquisition section
10 biological signal analysis section
11 control section
12 system bus

1. A diagnostic imaging apparatus comprising:
an imaging section obtaining an image of a site of an examinee and generating image data;
a display section displaying the generated image data;
a biological signal acquisition section acquiring biological signal data which is periodical movement of the site of the examinee;
a storage section synchronously storing the generated image data and the acquired biological signal data;
a biological signal analysis section analyzing the biological signal data to detect particular signal waveforms; and
a control section causing the biological signal analysis section to calculate evaluation values, on the basis of time difference or time ratio among a plurality of successive periods constituted by intervals among the detected particular signal waveforms, wherein the calculate evaluation values indicates steadiness of the periodical movement among the respective periods and to extract a detection period from among the plurality of successive periods on the basis of the calculated evaluation values and a time difference or time ratio threshold range, and reading out image data generated during the extracted detection period, among the image data stored in the storage section, from the storage section, and causing the display section to display the read-out image data.

2. The diagnostic imaging apparatus according to claim 1, wherein the control section causes the biological signal analysis section to extract the detection period on the basis of an evaluation value closest to “1” in the case of using the time ratio, and such an evaluation value that the absolute value of the time difference is the smallest in the case of using the time difference.

3. The diagnostic imaging apparatus according to claim 2, further comprising a first specification section specifying the number to be extracted as the detection periods, wherein
the control section causes the biological signal analysis section to extract the specified number of detection periods in order from the evaluation value closest to “1” in the case of using the time ratio and causes the biological signal analysis section to extract the specified number of detection periods in order from such an evaluation value that the absolute value of the time difference is the smallest in the case of using the time difference.

4. The diagnostic imaging apparatus according to claim 1, wherein the control section calculates a second evaluation value indicating deviation of the evaluation value of each period relative to a desirable first evaluation value that appears when the periodical movement is steady, and performs sort processing in ascending order of the second evaluation values of the respective periods to extract the detection periods in order from the highest position as a result of the sort processing or performs sort processing in descending order of the second evaluation values of the respective periods to cause the biological signal analysis section to extract the detection periods in order from the lowest position as a result of the sort processing.

5. The diagnostic imaging apparatus according to claim 1, wherein the control section causes the display section to display the detection period in a biological signal diagram showing the biological signal data being distinguished from other periods.

6. The diagnostic imaging apparatus according to claim 1, further comprising a second specification section specifying a range of evaluation values to be highlighted, wherein
the control section causes the display section to highlight a period showing an evaluation value included in the range of the evaluation values to be highlighted in a biological signal diagram showing the biological signal data.

7. The diagnostic imaging apparatus according to claim 3, wherein the control section displays images based on image data acquired during the detection periods in relatively small display areas and enlargedly displays an image based on image data during a detection period having the most favorable evaluation value in a relatively large display area of the display section.

8. The diagnostic imaging apparatus according to claim 1, wherein the control section causes the biological signal analysis section to compare the detection periods with predetermined first set time and second set time relatively longer than the first set time and to extract a detection period with time longer than the first set time and shorter than the second set time.

9. The diagnostic imaging apparatus according to claim 1, wherein the control section causes the biological signal analysis section to compare periods used for calculation of the evaluation values of the detection periods with predetermined first set time and second set time relatively longer than the first set time and to extract a detection period for which the evaluation value has been calculated with the use of only periods with time longer than the first set time and shorter than the second set time.

10. The diagnostic imaging apparatus according to claim 1, wherein the control section causes the biological signal analysis section to compare all periods of the biological signal data with predetermined first set time and second set time relatively longer than the first set time, and to extract only periods with time longer than the first set time and shorter than the second set time, and to extract the detection periods on the basis of a period constituted by at least three or more successive such periods.

11. The diagnostic imaging apparatus according to claim 1, further comprising a storage section synchronously storing the biological signal data and the image data, wherein
the control section causes the biological signal analysis section to extract the detection period on the basis of the biological signal data read from the storage section.

12. The diagnostic imaging apparatus according to claim 1, wherein:
the control section causes the biological signal analysis section to acquire the biological signal data from the biological signal acquisition section in real time and to perform extraction of the detection period;
causes the display section to display an image of the detection period; and
when having caused the biological signal analysis section to extract a new detection period, causes the display section to perform update display of an image picked up during the new detection period.

13. The diagnostic imaging apparatus according to claim 1, wherein the biological signal acquisition section acquires electrocardiographic data of the examinee;
further comprising an ultrasound probe transmitting ultrasonic waves to the examinee and also receiving a reflected wave to generate a reflected echo signal, an ultrasonic wave transmission/reception section transmitting a pulse signal for causing the ultrasound probe to radiate ultrasonic waves and also performing control for causing the ultrasound probe to acquire the reflected echo signal, and an ultrasound image generation section generating an ultrasound image on the basis of the reflected echo signal;
wherein the control section controls the ultrasonic wave transmission/reception section and the ultrasound image generation section, when the ultrasound probe is contacted with the site of the examinee, for obtaining an ultrasound image of the site of the examinee during the detection period; and displays the ultrasound image picked up during the detection period, on the display section.

14. The diagnostic imaging apparatus according to claim 1, wherein the control section causes the biological signal analysis section to calculate either the ratio or time difference of two successive periods adjoining a period to be an evaluation value calculation target as the evaluation value, to extract periods as detection periods in order from an evaluation value closest to 1 in the case of the time ratio, and to extract periods as detection periods in order from such an evaluation value that the absolute value of the time difference is closest to 0 in the case of the time difference.

15. An image display method comprising the steps of:
imaging an image of a site of an examinee to generate image data by an imaging section;
acquiring biological signal data which is periodical movement of the site of the examinee by a biological signal acquisition section;
synchronously storing the generated image data and the acquired biological signal data into a storage section;
analyzing the biological signal data to detect particular signal waveforms by a biological signal analysis section; and
calculating, on the basis of time difference or time ratio among a plurality of successive periods constituted by intervals among the detected particular signal waveforms, evaluation values indicating steadiness of the periodical movement among the respective periods, causing the biological signal analysis section to extract a detection period from among the plurality of successive periods on the basis of the calculated evaluation values and a time difference or time ratio threshold range, and reading out image data generated during the extracted detection period, among the image data stored in the storage section, from the storage section, by a control section; and
displaying the read-out image data on a display section.
